(19) United States
(12) Patent Application Publication
(10) Pub. No.: US 2009/0203606 A1
(43) Pub. Date: Aug. 13, 2009

(54) Systems for and Methods of use of Therapeutic Nutrition for the Management of Age-Associated and Age-Specific Health Conditions of the Elderly

(76) Inventors: Robert R. Wolfe, Little Rock, AR (US); John P. Troup, Plymouth, MN (US); Nicolaas E.P. Deutz, Little Rock, AR (US)

Correspondence Address: LEWIS, RICE & FINGERSH, LC;ATTN: BOX IP DEPT., 500 NORTH BROADWAY, SUITE 2000, ST LOUIS, MO, 63102, US

(21) Appl. No.: 12/366,520

(22) Filed: Feb. 5, 2009

Related U.S. Application Data
(60) Provisional application No. 61/026,302, filed on Feb. 5, 2008.

(51) Int. Cl. A61K 38/00

(52) U.S. Cl. 514/12

(57) ABSTRACT

Nutritional compositions which provide for improved taste profiles for the elderly while simultaneously providing nutrition specific to assist in the management of nutritional deficiencies that lead to age specific and age associated health conditions in the elderly. There are also provided products using these compositions and incorporating packaging design, volume delivery, and sensory attributes to improve nutritional regimen compliance in the elderly.

### CROSS REFERENCE TO RELATED APPLICATION(S)

This application claims the benefit of U.S. Provisional Patent Application Ser. No. 61/026,302, filed Feb. 5, 2008, the entire disclosure of which is herein incorporated by reference.

### BACKGROUND

1. Field of the Invention

The present invention relates to the methods and practice of nutritional supplementation to manage nutritional deficiencies that lead to age specific and age associated health conditions in the elderly, specifically the use of a combination of components specific to improving health status in the elderly, including optimal package design and volume delivery, carrier materials having specific sensory attributes, and specific nutrient blends.

2. Description of the Related Art

Health status during the aging process is significantly influenced by nutrition. Malnutrition has been shown to increase mortality in the aged and protein energy undernourishment has been shown to accelerate the age associated rate of muscle loss (sarcopenia), disease associated loss of muscle mass (cachexia), and to complicate the therapeutic management of cardiovascular disease, diabetes and other metabolic based disorders. With inadequate nutritional intake during aging, a shift toward an increased amount of body fat mass takes place and the ratio of muscle mass to fat mass decreases.

As a result, the aged individual often enters a hyper-inflamed state due to increased cytokine production from adipose tissue which complicates the management of the health status of the aged individual and increases the risk for the development of co-morbidities. Further, this state can prolong recovery from both acute and chronic conditions.

As a result of the strong association between health and nutrition, nutritional guidelines have been made available by a number of professional associations and government agencies. These guidelines, however, have almost universally simply emphasized caloric intake achieved through daily meals, supplementation or meal replacement without concern of the specific physiological needs and changes present due to aging. Thus, current nutritional practices and methods make no provision for the unique therapeutic requirements of the elderly, or the specific and targeted benefit that key nutrients provide in compromised health stages. Instead, they are focused on caloric intake and the sources of such calories (e.g., from vegetables vs. meats). These guidelines, therefore, have had limited effect on the improved health outcomes of the elderly as they are overly general and also do not focus on specific nutritional needs related to aging.

With nearly 50% of free living elderly suffering from health conditions caused or exacerbated due to poor nutrition, and over 40% (and even up to 70%) of hospital admissions of the elderly having been diagnosed as undernourished, optimizing health care and those factors influencing health outcomes should be an important consideration. It has been estimated that between 25% and 70% of institutionalized elderly patients are classified as malnourished and as many as one-half of this number are protein under-nourished. Therefore, implementation of strategies to promote healthy nutritional status in the elderly is critical. In an effort to optimize the role of nutrition with a focus on improving health status, a better use of nutrition practices and delivery is therefore desirable. Specifically, a need exists for an organized effort to optimize the role of nutrition in promoting health and to offer nutritional compositions in forms and as part of services to improve health outcomes in selected therapeutic areas.

In the last 15 to 20 years a growing number of scientific publications have described the benefits of improved nutrition, the role that specific nutrients play in targeting mechanisms and pathways of disease states. More specifically the benefit and need for greater protein intake with specialized amino acid profiles in the elderly (specifically those 50 years and older) has been shown to improve chronic and acute health conditions. Supporting studies have highlighted the fact that improving nutrition with increased protein intake alone in the elderly can attenuate muscle loss, facilitate recovery in trauma and post-surgical situations, and promote wound healing.

Effective nutritional support has also been shown to provide cost effective improvement in the care of the elderly. One estimate of the healthcare cost attributed to sarcopenia (in the U.S.) in 2000 was $18.5 billion and it was concluded that the initial treatment to reduce muscle loss is to ensure the intake of adequate protein. This information supports the role that age specialized nutritional support can also play a central role in cost effective patient care. Specifically to provide that hospitalized length of stays are decreased and recovery from typical age-associated conditions is enhanced during such stays.

The most prominent and consistent physiological change that occurs with aging is a loss of muscle mass and function that is termed “sarcopenia”, a condition which is not dependent on disease. The importance in addressing sarcopenia is clear in light of its correlation to functional impairment, disability, falls, frailty, and the loss of independence that increases with aging. In addition, sarcopenia has been linked to decreased bone density and impaired glucose tolerance. The etiology of sarcopenia includes decreased physical activity, malnutrition, increased inflammatory cytokine production, oxidative stress, and reductions in both growth hormone and androgens.

A variety of other changes with aging may in fact be secondary to changes in muscle Examples include a change in body composition, favoring a higher percent of body fat. Elderly with a greater fat mass are more likely to suffer from impaired glucose tolerance and diabetes. Impaired insulin sensitivity occurs in approximately 43% of individuals over 60, and this leads to frank diabetes in 16%. It has been reported that type 2 diabetes is a major health concern that affects nearly 20% of elderly adults, but the prevalence of diabetes in the elderly is not well established. Since insulin insensitivity occurs principally in muscle, the mechanism of development of insulin resistance with aging can be considered an aspect of progressive muscle dysfunction possibly related to reduction in mitochondrial numbers and metabolic function. Decreased bone density, osteoporosis and increased susceptibility to fracture commonly occur with aging, particularly in women, but also in men. Decreased muscle strength and function may relate to impaired bone health, given the relation between the torque placed on bone by muscle contraction and the strengthening of bone. The elderly suffer from reduced immune status and altered immune function and chronic inflammation have been claimed by some to underlie catabolic changes in muscle leading to sarcopenia and in turn increasing the need for protein to increase anabolism.

Aging is associated with a decrease in skeletal muscle mass and an increase in body fat. Because a majority of the inflammatory cytokines are derived from adipocytes it would reason that an increase in the proportion of fat to lean body mass would increase the risk for a heightened inflammatory response. It has been proposed that loss of muscle mass results in a condition called ‘sarcopenic obesity’ as originally observed in elderly with rheumatoid arthritis and osteoarthritis. Research indicates that the inflammatory cytokines produced by adipose tissue, especially visceral fat, accelerate muscle catabolism and thus contribute to the vicious cycle that both initiates and sustains sarcopenic obesity. Subjects with sarcopenic obesity at baseline were two to three times more likely to report disability than lean sarcopenic or non-sarcopenic obese subjects and those with normal body composition. As lean muscle is lost, the body's proportion of fat increases, cytokines that promote skeletal muscle catabolism also increase. Therefore, improved lean mass or attenuation of sarcopenia through an increased dietary protein intake reduces sarcopenic obesity and its associated rise in inflammatory cytokines.

While physiological and metabolic changes of aging can be influenced with optimal nutritional support, current methods and practices have not provided the level of specialization required to provide for effective nutritional management. Nutritional practices in use today call for the provision of 3 meal servings per day designed to deliver total daily caloric requirements. Aging however, results in a number of changes to the human body that make this delivery approach generally ineffective in the elderly. First, nutrient deficiencies of some amino acids (i.e., arginine) are known to lead to stomach constriction early in meal consumption such that early satiation is achieved and fewer calories are consumed. This contributes to a 20 to 30% decrease in caloric consumption in the aged (65+ yr vs. 40− yr), a gradual 30% decrease in energy cost and at least a 15% deficit in required caloric intake. Most effected by this profile is a significant reduction in protein intake with aging and a shift in total fat away from healthy fats (i.e., poly and mono unsaturated fats) to fats associated with increased incidence of compromised health status (i.e., saturated and trans fats). With this practice alone, nutritional support in the traditional sense, therefore, has no therapeutic benefit to the elderly consumer as the specific disease needs are not being targeted with the active components that nutrition can provide.

In some cases, the above limitation has been addressed through the use of meal supplementation and/or meal replacement. With the observation that fewer calories per meal setting are consumed by the elderly, nutritional supplementation has been provided with the meal in attempt to increase total nutritional intake. Nutritional supplementation has generally taken the form of nutritional beverages. These beverages are in a high milk-fat based format often with over 250 calories provided in 8 ounce servings. In the case where a meal is not consumed, these same beverages could also be provided as replacement to the meal and so as to provide an alternative nutrient intake. By providing a high calorie supplement of particular nutritional profile, the intention is to provide for both increased caloric consumption and specific nutritional supplementation.

The effectiveness of these methods have been limited for a number of reasons. First, the beverages are generally not fully consumed (less than 60% volume consumed has been reported) and compliance after 3 weeks of use falls to below 50% of recommended use. This poor outcome is in part due to unacceptable levels of sensory acceptance as the taste profiles of the elderly are significantly altered with a higher sensitivity to bitterness and aversion to the taste of intact milk protein and the vitamin or mineral like taste of the product. Thus, the net effect is substitution rather than supplementation and no improvement in therapeutic support generally takes place.

To further compensate for problems in nutritional intake, caloric dense products have also been developed for use as supplements. The nutrient profiles of these products, however, do not address the specific therapeutic needs any more than the other beverages. They do never-the-less makeup some of the deficit in caloric intake observed with other products, but because of the amount of calories provided in the same volume, less liquid is available which in turn compromises hydration of the consumer which is also a major issue in the elderly.

### SUMMARY

Given the age specific requirements of the elderly consumer, their therapeutic specific nutritional support needs combined with the limited to neutral effect of existing nutritional methods and practices, a need for effective therapeutic specific nutritional support is needed.

Discussed herein is a nutritional regimen and associated nutritional supplement products which are designed specifically to appeal to elderly (50+ years in age) consumers. The products are designed to target specific deficiencies, such as protein deficiencies, seen in the elderly, are provided in a more effective carrier than prior milk-based supplements, and provide for taste profile more acceptable to elderly tastes.

There is described in a composition comprising: a component including: intact protein; and at least one essential amino acid selected from the group consisting of leucine, isoleucine, valine, lysine, methionine, histidine, phenylalanine, theonine, and tryptophan; and a carrier component comprising a fruit juice.

In an embodiment, the carrier component may comprise a combination of fruit juices which may be selected from the following combinations: loquat and lime, mango and peach, black cherry and apple, pomegranate and cranberry, plum and tea, apple and peach, blueberry and grape, cranberry and lemon, orange and naranjilla, or pineapple and naranjilla.

In an embodiment, the composition further comprises a low-glycemic carbohydrate, an omega-3 fatty acid, arginine and/or citrulline.

There is also described herein a drink product comprising: a liquid solution comprising: protein; at least one essential amino acid; and a fruit juice; and a bottle for holding said liquid solution, said bottle having: a top; a neck arranged toward said top; and a bottom having a larger circumference than said top.

In an embodiment, the bottle holds between about 8 and about 12 ounces of liquid.

### BRIEF DESCRIPTION OF THE FIGURES

FIG. 1 provides an embodiment of a bottle suitable for use in supplement delivery.

FIG. 2 shows the effects of aging on taste and smell.

FIG. 3 illustrates flavor characteristics compared with sensations of hunger.

FIG. 4 illustrates the effect of product form and timing of intake.

FIG. 5 shows a chart indicate servings per week preferences on Juice vs. Milk based products.

FIG. 6 illustrates that the use of essential amino acids more effectively improves protein synthetic rates in the elderly.

FIGS. 7 and 8 illustrate the difference that exists in aging vs. younger consumers.

FIG. 9 indicates that in combination with higher levels of essential amino acids leucine provides a greater stimulating effect on protein synthetic rates in the elderly.

FIG. 10 presents a comparison of effect of different blends to achieve total protein availability.

FIG. 11 illustrates the superior functional improvement effect in the elderly when the specialized protein source blend of essential amino acids are used vs. standard product.

FIG. 12 shows the glycemic index of specialized blends of protein and slow release carbohydrate beverages vs. standard nutritional beverages.

FIG. 13 indicates that managing protein synthesis when omega 3 fatty acids are combined with protein sources can provide greater rates of protein synthesis.

FIG. 14 presents data describing the use of different combinations on the ability to increase protein synthesis rates.

FIG. 15 illustrates improvement in functional limitation in an elderly hyper-inflamed group.

FIG. 16 provides additional support that therapeutic nutrition is an effective treatment modality.

FIGS. 17 and 18 present the improved outcome as a result of combining therapeutic modalities of exercise and specific nutritional support.

### DESCRIPTION OF PREFERRED EMBODIMENT(S)

Disease specific nutrition or nutrition provided to have therapeutic purpose is an important adjunctive care regimen for the management of age specific and age associated health. The method of age-specific health management via the method and approach of such nutritional regimens differs from nutritional support methods historically implemented. Age specific changes compromising health are preferably managed with the use of very specific nutrient compositions as opposed to a general approach where one nutritional formula is used for all consumer groups.

Specifically, nutritional formulas or blends are chosen to correspond to the needs and requirements of elderly populations instead of selecting formulas designed for use by persons of any age. For purposes of this disclosure, a human is defined as “elderly” if they are at least 50 years of age. In an embodiment, therefore, the supplements and regimens discussed herein are intended for use with a human of 50 years of age or more. In an alternative embodiment, they are for use with a human of age 60 or more, and in a still further embodiment may be used for those aged 65 or more.

This new method of therapeutic targeted nutritional support is based on the selection of combinations in a nutrient based formula that addresses the specific basis of the aging process and geriatric disease states. As discussed above, the elderly consumer and patient has very different physiological and metabolic differences and requirements when compared to one in a younger population. Therefore, there are described herein targeted and specialized nutritional solutions designed to provide improvement in health outcomes for the elderly. Further, this method also defines components and compositions including such formulations that improve compliance within an elderly population by taking into account preferred timing of consumption, volume available in a specific product presentation or serving, product form, and type and sensory profile of a carrier material and the resultant supplement. Any or all of these components may then be used in a successful care regimen that improves effectiveness of supplementation.

The management of hyper-inflammation and need for optimized protein effect will generally result in improved physical function of the elderly using these specific nutritional methods. Disclosed herein are methods of use for nutritional support that increase muscle protein mass, strength and physical function in the elderly, including but not limited to individuals over 60 years of age. The compositions generally comprise total protein and higher levels of selected amino acids (as described below) achieved by using a blend of amino acids, peptides high in leucine, intact protein sources, slow release long chain saccharides/carbohydrates in which the proportion of each amino acid and the amount of carbohydrates and/or protein are tailored to optimize muscle protein synthesis and muscle protein turnover, and consequently increase muscle mass, strength and physical function.

Effective use of nutrition for age-associated conditions in the elderly is defined by the combined use of three major components in combination. Firstly, the nutritional supplement is providing in a packaging design which provides convenience and ease of use (form) and delivery (volume) of the active nutrient blend in such a way that the package is ergometrically designed specific to an elderly population while still being able to deliver the active components of the nutritional formula. This generally provides for an improved level of compliance in a nutritional regimen.

Secondly, the carrier substrate in which the nutritional blend is provided within the packaging design is selected to improve compliance by providing a taste profile which is generally more desirable for an older consumer. Specifically, the carrier is provided for oral consumption of the nutritional formula and will generally be in a liquid fruit or vegetable juice form with high sensory profile of low bitter and higher sweetness attributes. Flavors are enhanced and designed to meet this criteria often by blending of juice flavors. This is also designed to improve compliance with nutritional regimens and also provide for product preference over potential substitute products which lack the nutritional supplementation.

Thirdly, the blend formula, either taken alone or in combination with components of the carrier, is designed to target specific metabolic and physiological pathways and systems in the elderly population. The nutritional formula is therefore designed to deliver an age-specific and therapeutic-specific blend of key macronutrients which are intended to act on the age specific mechanisms of the therapeutic state and thus enhances the aged specific outcome and effect. The method of designing and providing such formulas includes selection of nutritional components affecting health conditions influenced by protein metabolic pathways and control systems, inflammatory response and control and insulin resistance.

In addition to including specific nutritional formulations products, in an embodiment, the nutritional product may also comprise a part of a larger consumer health management program for exercise and nutrition which may include the use of nutrient-drug co-therapies in management of sarcopenia, cachexia, diabetes, cardiovascular health and other health conditions of high incidence specific to the elderly.

In order to provide for a discussion of nutritional formulas, carriers, packaging, and other elements of a care program for the elderly, it is desirable to look at individual components and then at the interaction of the components as a whole.

### Packaging Design

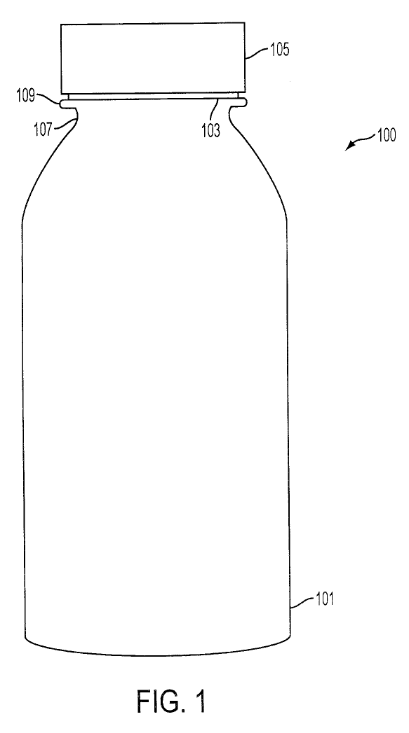

Ergometric evaluation has determined preferred bottle circumference and form to fit the hand grip of elderly individuals. Additionally, pulsating grip closure requires neck like shapes of bottles to avoid slippage of the bottle in the hands. Further, to compensate for the unsteady placement of bottles when they are set down, the base of the bottle is preferably larger than the top and supports greater stability. To provide for these benefits, FIG. 1 provides for an embodiment of a bottle in which a liquid supplement of the present discussion may be provided. In the embodiment of FIG. 1 there is provided a bottle (100) which is designed to have a wider stable bottom portion (101) and a narrower mouth (103). The bottle (100) is closed by a cap (105) which may be resealable. The bottle (100) also includes a narrow neck (107). In the depicted embodiment, the neck terminates in a disk (109) prior to the mouth (103) which can also help prevent slippage.

Depending on embodiment, the bottle is preferably made of plastic, but may alternatively be made of glass or other materials. Further, the container in FIG. 1 is shown with smooth sides but may alternatively include ribs, indents, extensions, or other shapes to improve grip and to make it easier to hold onto the bottle as is known to those of ordinary skill.

Volume availability per serving is a factor in achieving compliance in use of supplementation and to encourage snack occasions. Specifically, individual servings which are too large will not be fully consumed resulting in the nutritional formula not being completely consumed in a consumption event and the nutritional benefit not being correctly applied. Similarly, servings which are too small can result in over consumption or substitution. In an embodiment it is preferred that the bottle hold about 8-12 ounces of supplement when filled. Data has shown that over a 12 week period of supplementation use where about 8 to 12 ounces per serving were available, 80% of users consumed between about 4 and about 6 ounces (about 100 to about 180 ml) of the nutritional beverage. Volume of delivery of the liquid beverage for optimal intake and compliance is therefore preferably about 170 ml with a range of about 100 to about 180 ml. Compliance is further improved by the specialized bottle design making consumption easier.

While size and presentation influence consumption, intended timing of product use is also an important part of the methodology involved in providing for nutrition with therapeutic benefit. Since the effective use of this methodology is specific and improved outcomes for the elderly, use of this regimen is based on first: the ability to optimize and stimulate the preferred outcome that the product offers and second: to further enhance this effect, provide the product at times and in manner that maximizes the effect of this first point. To this end, in an embodiment the product is designed to be consumed between meals. When used at this time, the formulations exert an improved stimulation of protein synthetic pathways. Alternatively, the product can also be used at meal time as it enhances the stimulatory effect of the protein in the meal on protein synthesis.

The use of the product between meals is generally described as ‘active snacking’ and as such provides opportunities in the mid morning, mid afternoon and early evening to use the product in order to stimulate protein synthesis. With this approach, the ability to build muscle mass and improve muscle function is enhanced as the more frequent the stimulation, the greater the effect.

Use between meals has also been shown to be effective as part of total patient care and co-therapy in total health management. An important component in the method of use of the nutritional formulas is the combined use where the effect has complimentary and synergistic effect. Combination of this method and product as part of a planned long term exercise program has also been shown to increase protein synthesis and optimize the effect and benefit on management of the muscle wasting conditions of sarcopenia and cachexias well as the consequence of recovery from hip fracture or chronic hospitalized care. Similarly, when used in combination with drug therapy, complimentary and synergistic effects are possible.

### Taste & Flavor

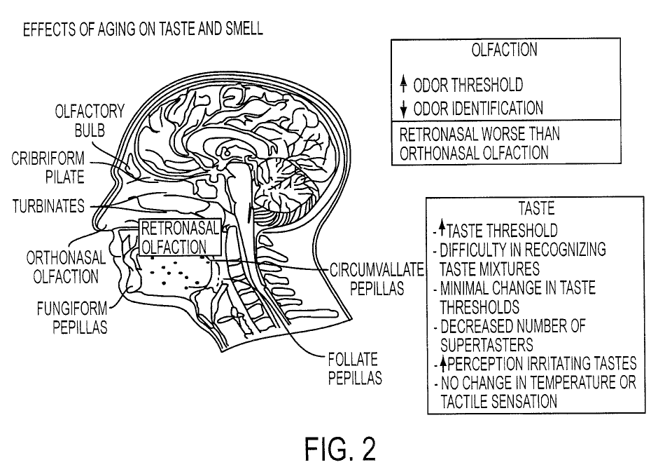

Taste perceptions significantly change with aging and lead to a lack of interest in food and thus lower caloric intake. This change in sensory capability results in decreased compliance in the use of products both for continuous daily use and per serving consumption volume. The basis of this effect and alterations are illustrated in FIG. 2. As one ages generally the sensation of bitterness is increased and sweetness is decreased. Olfactory changes also take place further diminishing the palatability of food. These changes require that supplements must contain robust flavors and smells if compliance can be improved.

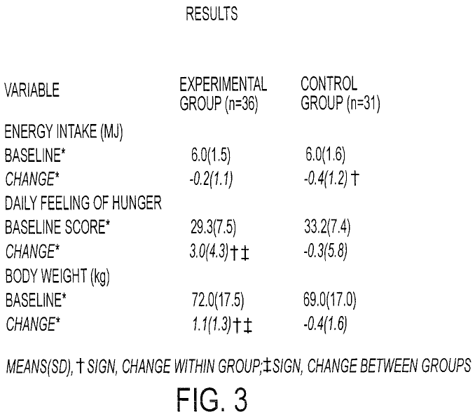

The effectiveness of flavor enhancement was tested in a group of elderly volunteers using supplements with and without flavor enhancers. As shown in FIG. 3, the experimental group with robust flavor enhancement and lower sensation of hunger, complied with daily dietary regimen and achieved greater weight gain.

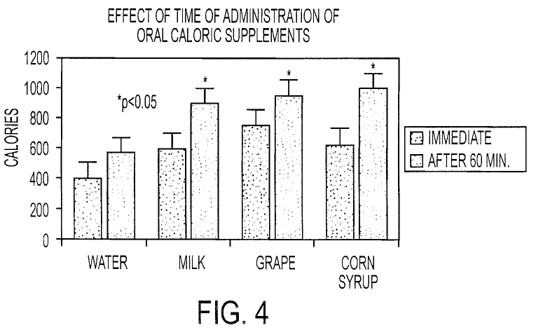

FIG. 4 illustrates the effect of product form and timing of intake. This data shows that supplementation at least 60 min after a meal will result in long term more caloric intake and suggests that active snacking and between meal supplementation is more effective to weight gain in the elderly as contemplated earlier. Additionally, when comparing product forms of milk (the most typical form of supplementation today) with grape flavored juice, the data illustrates juice form of products was more effective at weight gain and consistent intake of calories and confirm that robust flavored juice beverages are preferred over milk type beverages.

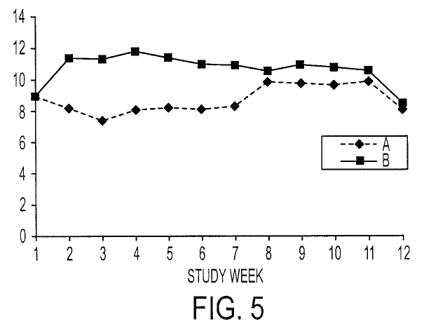

Furthermore, compliance in total number of servings/week (FIG. 5) of milk based products was 57% of the total desired amount to be consumed while that of juice based nutritional support was 86%. Benchmark taste comparisons also reveal a 2:1 preference toward juice based product use vs. milk based product which also accounts for selection and higher compliance data. FIG. 5 illustrates number of servings consumed per week with a milk based format vs. groups using juice based drink formats. Compliance with juice based drinks are superior to milk based drinks.

Preferably, the selection and use of flavors will be to provide those that are not averse and are preferred by the elderly. Flavor selection will preferably have low to no bitterness and higher and more robust flavor attributes such as sweetness. These flavor profiles are achieved in an embodiment by use of fruit juices as a carrier liquid for the nutritional supplement. Further, the flavors may be enhanced in an embodiment by using fruit combinations. Some such desired combinations include, but are not limited to, loquat and lime, mango and peach, black cherry and apple, pomegranate and cranberry, plum and tea, apple and peach, blueberry and grape, cranberry and lemon, orange and naranjilla, and pineapple and naranjilla. These flavors are preferably provided in clear or nearly clear juice liquid forms with no milky residue or pulp, achieved by using high acid treatment of whey protein isolates (pH less than 3.0 and range of 2.0 to 5.0). Juices may be natural, may comprise artificially flavored materials, or may be a combination of the two.

### Nutrient Formula Blend and Nutrient Selection

Over the last 20 years, mounting scientific evidence has provided new information on the role and impact that nutritional status has on the functional capacity and health status of the aging individual. A number of studies have pointed to protein and selected fats as key nutrients in the elderly. Protein intake greater than the amount required to avoid deficiency can ameliorate chronic wasting (i.e., loss of muscle mass) associated with the aging process. With a 20% loss in muscle mass possible between the ages of 50 and 60 years, the ability to limit this negative cascade and avoid compromised health status is important. Other tissues, organs and physiological processes can also potentially benefit from increased protein intake. The benefits of increased protein intake may be reflected not only in improved function and quality of life in the healthy elderly, but also the ability of hospitalized elderly patients to recover from disease and trauma such that health outcomes are improved and cost of care is decreased.

One study suggested that inadequate consumption of protein was highly correlated with elevated levels of inflammatory mediators. Oxidative stress and sub-acute diseases that promote inflammatory mediators (i.e., cytokines) are associated with muscle loss in the aging process. Cytokines are correlated with both diabetes and diminished lean muscle mass in the elderly, these include tumor necrosis factor (TNF-α), interleukin-6 (IL-6), and C-reactive protein (CRP). The importance of chronic inflammation on muscle loss is emphasized by its association with aging and disease as the primary cytokines (e.g., IL-6, IL-1β, and CRP) are highly correlated to increased rates of functional disability, muscle degradation, and mortality in the elderly. In one study, higher IL-6 and TNF-α production was associated with reduced muscle strength and increased mortality. Thus, with an increase in the catabolic processes imaging, driven by a hyper-inflamed state, an increased need and use of protein may help achieve a more positive balance in protein synthetic rates and slow the loss of muscle mass.

The ability to build muscle decreases with age by up to 30% and thus the ability to maintain rates of muscle synthesis in aging require elevated protein intake. This is an important issue in aging as the amount of muscle mass loss per year is on average 2%, thus over a decade the muscle mass loss approaches on average 20% of weight and can result in a significant loss of function. A further complication is the increasing levels of body fat seen in the elderly (50 years and older) resulting from the loss of muscle and concomitant shift to fat stores. The increased adipose tissue is a site of cytokine production which increases inflammation (with increased levels of IL6, TNF-a and CRP) and compromises immune system response while increasing risk of disease states.

While an increased protein intake will improve protein synthetic rates, in an embodiment the nutritional blend used herein utilizes a more potent form and source of proteins and amino acids through which even higher rates of protein synthesis can be achieved than with conventional intact proteins used alone. This is important since the hyper-inflamed state of the elderly increases the catabolic effect on muscle making anabolic processes more difficult to otherwise overcome.

Specific inflammatory therapeutic targets are important for the elderly since in addition to increasing muscle wasting in aging, TNF-α has been shown to modulate glucose metabolism and induce insulin resistance and down-regulate insulin receptor signaling in skeletal muscle. Increased TNF-α was reportedly associated with insulin resistance in age-associated muscle wasting and has been shown to be elevated in older men and women and in the skeletal muscle of obese and diabetic humans. Conversely, IGF1 levels were shown to be decreased in aging individuals which further decrease protein synthetic rates and accentuate the loss of muscle mass leading to cachexia and sarcopenia. Recent studies that the intake of elevated protein increases IGF1 levels which in other studies have resulted in a 15% reduction of adipocity and a 10% increase in muscle mass. Further, elevated protein intake has also resulted in improved blood glucose control along with a concomitant improvement in an index of insulin resistance. Thus, these data suggest that while the aging process leads to negative changes in inflammatory modulators and muscle growth factors, increased protein intake plays an important role in minimizing these age associated issues and our method of incorporating a more potent form of total protein is more effective at controlling these age specific responses.

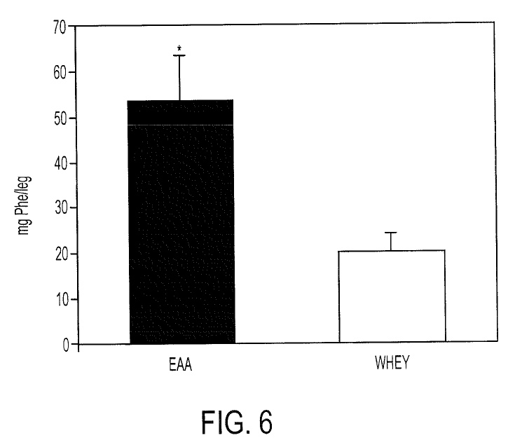

In an embodiment, protein is provided in the nutritional formula as a blend enriched with the essential amino acids (EAAs) that cannot be synthesized in adequate quantities in the body. These include, but are not limited to, leucine, isoleucine, valine, lysine, methionine, histidine, phenylalanine, theonine and tryptophan. FIG. 6 illustrates that intact protein alone is not as effective at stimulating muscle protein synthesis in the elderly as a mixture of EAAs with or without intact protein.

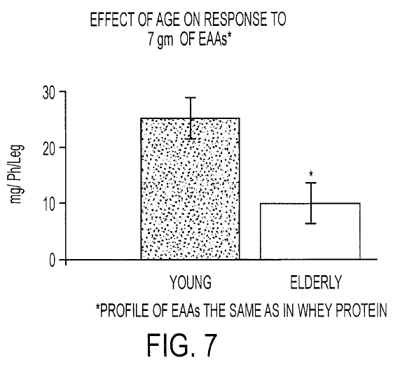

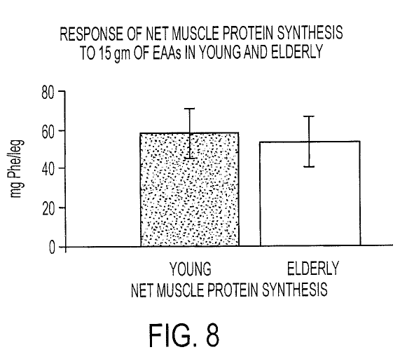

FIG. 7 illustrates the difference that exists in aging vs. younger consumers in response to a sub maximal does of EAAs. This illustrates why an age specific consideration in a blend in total protein is desirable. Clearly not only is the type of available protein/amino acids of interest but consideration of total available amount is also desirable and described in the methods and formulas contemplated herein. This need and result is presented in FIG. 8 which illustrates that if intake of the same mixture of EAAs shown in FIG. 7 is sufficient, the response of muscle protein synthesis in elderly can equal that observed in younger counterparts.

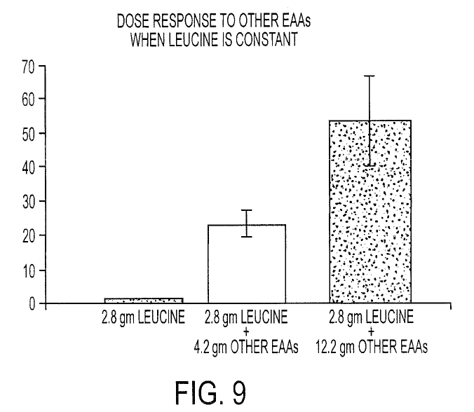

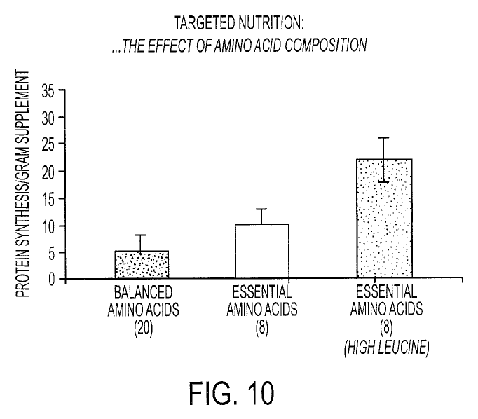

Since an increased potency of the protein effect is desirable to the elderly, in an embodiment, the nutritional formula calls for the use and incorporation of at least one EAA and preferably the amino acid leucine as a part of the total source of proteins and amino acids. FIG. 9 presents resulting data and describes that in combination with higher levels of essential amino acids leucine provides a greater stimulating effect on protein synthetic rates in the elderly. FIG. 10 presents a comparison of effect of different blends to achieve total protein availability and highlights the preference for specialized amino acids and specifically leucine to assist the elderly in achieving higher levels of muscle protein synthesis. Due to these results, in an embodiment the nutritional blend used in the present supplement comprises at least 7 grams of leucine per 180 ml of total supplement or alternatively at least about 5% leucine in the supplement blend. In an alternative embodiment greater amounts of leucine may be used and may comprise at least about 10% leucine, at least about 15% leucine, or at least about 20% leucine. The formula will also generally include other EAAs and intact protein to provide for synergetic benefits as shown in FIG. 9. This blend will then be provided in the fruit juice carrier discussed earlier to provide for the supplement.

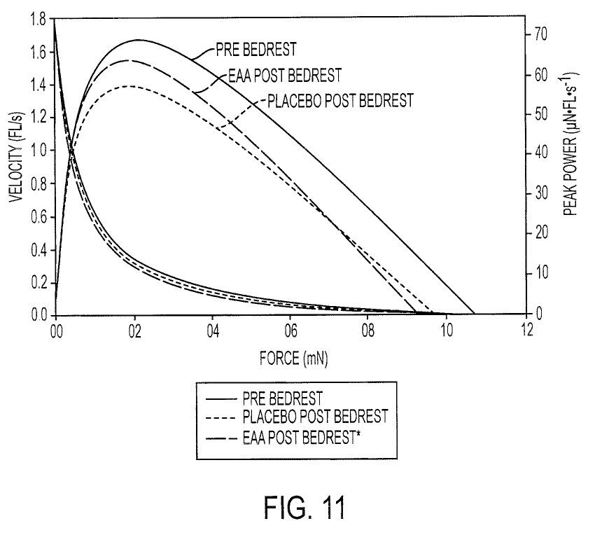

FIG. 11 illustrates the superior functional improvement effect in the elderly when the specialized protein source blend of essential amino acids are used vs. standard product forms not incorporating such nutritional formulas.

### Alternative and Additional Formulas

In addition to the protein and EAA blends discussed above, additional nutrient combinations can be useful for metabolic control and improved outcomes in the elderly. In a still further embodiment, to the nutrient formulas, blends, and compositions described above, there may also be added an active compound that further addresses and improves the benefit of the core formulation. The additional nutrient can include any of the following in any combination: creatine, ribose, theanine, omega-3 fatty acids, arginine, citrulline, cocoa-flavanols and lutein. The level of use of each these should range from 0.01 to 1 times that of the total amount of protein available in the formula.

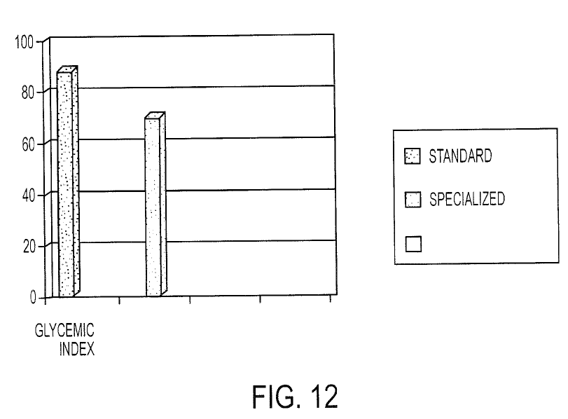

Due to the insulin resistance commonly observed in the elderly, protein metabolic pathways may be compromised such that the effectiveness of protein synthesis may be reduced. Thus, the insulin response may be influenced to yield more effective control of insulin and response to this anabolic hormone. To this end a blend of low glycemic carbohydrates may be used in combination with the protein profiles described above in an embodiment. This source and blend of carbohydrates not only provides the necessary energy for protein synthesis but also has improved insulin control compensating for the limitations of this anabolic hormone in the elderly. FIG. 12 presents the glycemic response of the carbohydrates in this method approach to elderly care.

Because inflammation causes catabolic process, it may also be important that the level of inflammation be controlled. The effect of individual amino acids on protein synthesis can be limited under hyper inflamed conditions and that as inflammation is controlled protein synthesis can increase. The use of omega 3 fatty acids and selected amino acids of arginine and citrulline can help reduce the inflammatory markers of IL6 and TNF-a thus allowing for a more effective anabolic processes.

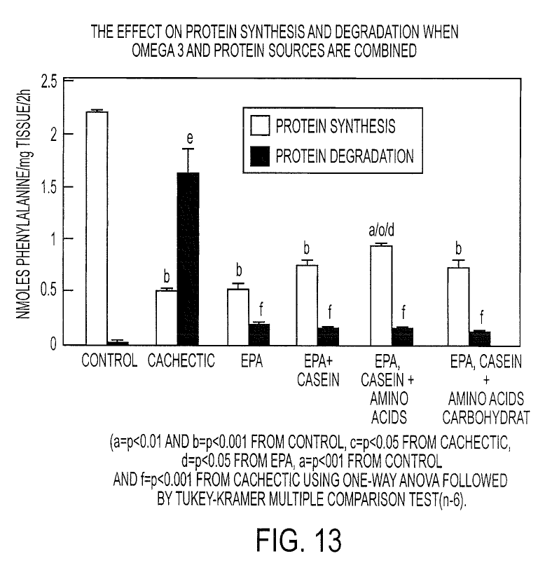

FIG. 13 provides that through managing protein synthesis when omega 3 fatty acids are combined with protein sources, greater rates of protein synthesis are achievable under cachetic conditions. This is the result of the ability of omega 3's to reduce the level of cytokines that are produced and cause the hyper-inflamed state. This data provides further support for the method of combined use of specific and select nutrients are important for therapeutic nutrition.

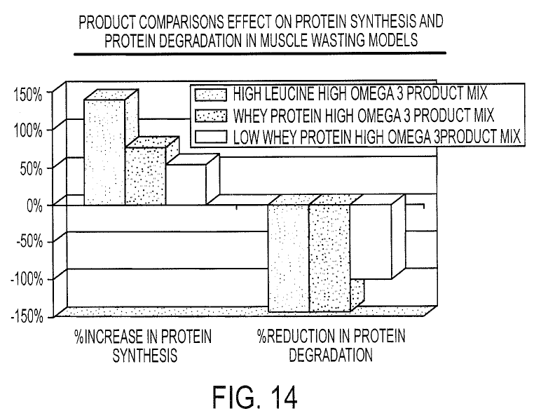

FIG. 14 presents data describing the use of finished products on the ability to increase protein synthesis rates where combination of protein types and the anti-inflammatory omega 3 fatty acids are available at different levels. When the preferred source and type of amino acid/proteins are used in combination with omega 3's a significant improvement is observed in anabolic response in elderly patients with muscle wasting taking place.

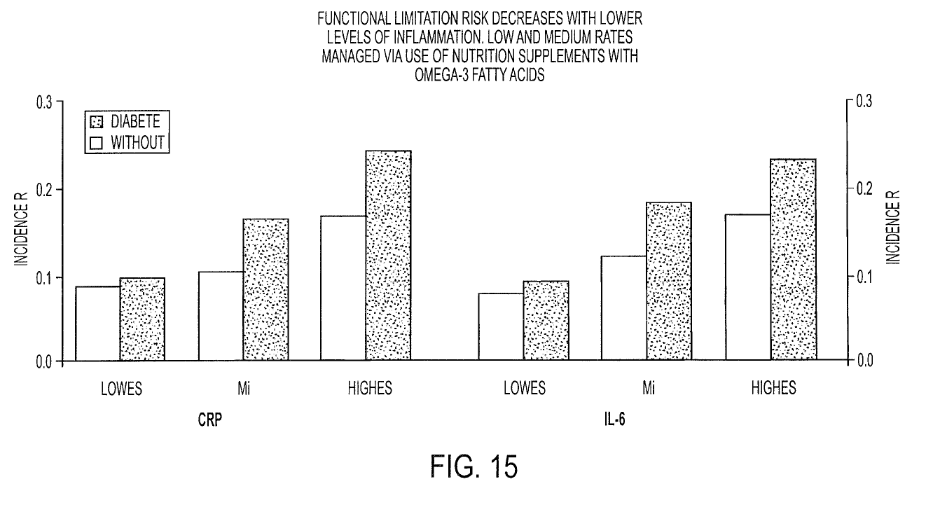

FIG. 15 illustrates the improvement in functional limitation in an elderly hyper-inflamed group of consumers. When therapeutic nutrition supplemental use is incorporated into the daily diet, inflammatory levels are decreased and the functional capabilities are improved.

In alternative or further embodiments, supplemental minerals may also be included. Suitable minerals may include one or more minerals or mineral sources with a focus on use of critical vitamins or minerals associated with benefit in the aging process. These include vitamin D, calcium, the family of B vitamins, vitamin A, E and C. Non-limiting examples of minerals include, without limitation: chloride, sodium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

The compositions may also optionally comprise vitamins. The vitamins may be fat-soluble or water soluble vitamins. Suitable vitamins include but are not limited to vitamin C, vitamin A, vitamin E, vitamin B12, vitamin K, riboflavin, niacin, vitamin D, vitamin B6, folic acid, pyridoxine, thiamine, pantothenic acid, and biotin. The form of the vitamin may include salts of the vitamin, derivatives of the vitamin, compounds having the same or similar activity of a vitamin, and metabolites of a vitamin.

The composition may also comprise at least one excipient. Non-limiting examples of suitable excipients include a buffering agent, a preservative, a stabilizer, a binder, a compaction agent, a lubricant, a dispersion enhancer, a disintegration agent, a flavoring agent, a sweetener, a coloring agent, and combinations of any of these agents.

In an embodiment, the excipient is a buffering agent. Non-limiting examples of suitable buffering agents include sodium citrate, magnesium carbonate, magnesium bicarbonate, calcium carbonate, and calcium bicarbonate.

The excipient may comprise a preservative. Suitable examples of preservatives include antioxidants, such as alpha-tocopherol or ascorbate, and antimicrobials, such as parabens, chlorobutanol, or phenol.

In an alternative or further embodiment, the excipient may be a binder. Suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosacchar ides, polypeptides, oligopeptides, and combinations thereof.

In an alternative or further embodiment, the excipient may be a lubricant. Suitable non-limiting examples of lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

The excipient may be a dispersion enhancer. Suitable dispersants may include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In yet another embodiment, the excipient may be a disintegrant. The disintegrant may be a non-effervescent disintegrant. Suitable examples of non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. The disintegrant may be an effervescent disintegrant. Suitable effervescent disintegrants include sodium bicarbonate in combination with citric acid, and sodium bicarbonate in combination with tartaric acid.

In another embodiment, the excipient may include a sweetener. By way of non-limiting example, the sweetener may be selected from glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose, and sugar alcohols such as sorbitol, mannitol, sylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), sucralose and sodium and calcium salts thereof.

It may be desirable to provide a coloring agent. Suitable color additives include food, drug and cosmetic colors (FD&C), drug and cosmetic colors (D&C), or external drug and cosmetic colors (Ext. D&C). These colors or dyes, along with their corresponding lakes, and certain natural and derived colorants, may be suitable for use in certain embodiments.

The weight fraction of the excipient or combination of excipients in the formulation may be about 30% or less, about 25% or less, about 20% or less, about 15% or less, about 10% or less, about 5% or less, about 2%, or about 1% or less of the total weight of the amino acid composition.

### Use of Nutritional Formulas and Supplements

The rationale underlying the compositions disclosed herein relates to both the individual effects of the components and their interactive effects. The amino acids will stimulate muscle protein synthesis. The particular essential amino acids are provided because they cannot be produced in the body and are thus their availability is rate-limiting for protein synthesis. The low glycemic carbohydrate will provide the energy needed to produce the new protein without eliciting a significant insulin response. The elderly are generally resistant to the action of insulin, so avoiding an insulin response by using low glycemic carbohydrate will be advantageous to that population. These selected carbohydrates are typically medium and long chain polysaccharides that are metabolically slow to digest and release low levels of glucose into the blood stream after digestion. These carbohydrates are typically classified as dextrans, multi-dextrans having more than 8 carbon units or chain lengths; commercial names include Nutriose, Sucramalt and others. Ribose may increase the amount of tRNA, which will be useful when combined with increased availability of the rate-limiting amino acids provided in this embodiment. The metabolic pathways by which glycine and ribose may stimulate RNA synthesis are shown in FIG. 14. Creatine increases muscle mass and improves strength by increasing energy stores in the muscle. When such components are coupled together, muscle strength, mass, and physical function will be increased to a greater extent than predicted by the response to any one component.

The method of providing total nutritional support includes the appropriate selection of each nutrient and blend. Amino acids may be included in the free form; in combinations of peptides; in combinations of intact protein and free amino acids; in combinations of free amino and peptides; or in combinations of free amino acids, peptides, and proteins. Low glycemic carbohydrate may comprise about 0-50% by weight.

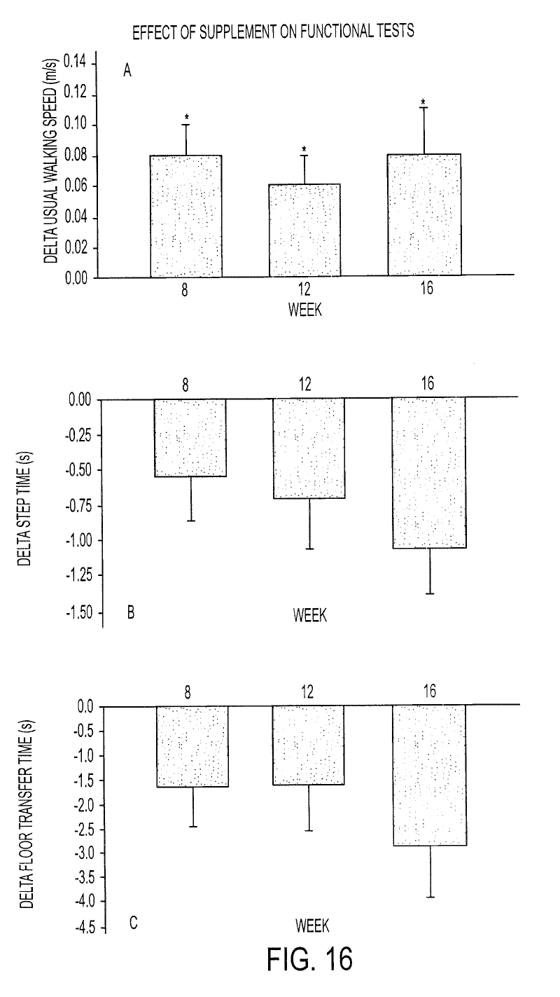

FIG. 16 provides additional support that the method of use of supplements such as those described herein is an effective treatment modality when used over a 12 week period for improved health management of age-specific and age-associated conditions. While this method defines the selection and use of key nutrients and combinations of nutrients that improve specific health outcomes, the method also provides for improving compliance and use of nutritional support systems through an ‘active snacking’ concept as the supplementation regimen where volume, taste and timing are critical to the effective use of this nutritional support method.

### Alternative Methods of Delivery

While it is preferred that the nutritional formula be provided in a fruit juice carrier, it is recognized that alternative methods of delivery can also be used. The nutritional formulas discussed herein may be formulated into a variety of forms and administered by a number of different means. The compositions may be administered orally, rectally, or parenterally, in formulations containing conventionally acceptable carriers, adjuvants, and vehicles as desired. The term “parenteral” as used herein includes subcutaneous, intravenous, intramuscular, or intrasternal injection, or infusion techniques. In an exemplary embodiment, the compounds of the invention are administered orally.

Solid dosage forms for oral administration may include capsules, tablets, caplets, pills, troches, lozenges, powders, and granules. A capsule typically comprises a core material comprising a composition of the invention and a shell wall that encapsulates the core material. The core material may be solid, liquid, or an emulsion. The shell wall material may comprise soft gelatin, hard gelatin, or a polymer. Suitable polymers include, but are not limited to: cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, preferably formed from acrylic acid, methacrylic acid, methyl acrylate, ammonio methylacrylate, ethyl acrylate, methyl methacrylate and/or ethyl methacrylate (e.g., those copolymers sold under the trade name “Eudragit”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). Some such polymers may also function as taste-masking agents.

Tablets, pills, and the like may be compressed, multiply compressed, multiply layered, and/or coated. The coating may be single or multiple. In one embodiment, the coating material may comprise a polysaccharide or a mixture of saccharides and glycoproteins extracted from a plant, fungus, or microbe. Non-limiting examples include corn starch, wheat starch, potato starch, tapioca starch, cellulose, hemicellulose, dextrans, maltodextrin, cyclodextrins, insulins, pectin, mannans, gum arabic, locust bean gum, mesquite gum, guar gum, gum karaya, gum ghatti, tragacanth gum, funori, carrageenans, agar, alginates, chitosans, or gellan gum. In another embodiment, the coating material may comprise a protein. Suitable proteins include, but are not limited to, gelatin, casein, collagen, whey proteins, soy proteins, rice protein, and corn proteins. In an alternate embodiment, the coating material may comprise a fat or oil, and in particular, a high temperature melting fat or oil. The fat or oil may be hydrogenated or partially hydrogenated, and preferably is derived from a plant. The fat or oil may comprise glycerides, free fatty acids, fatty acid esters, or a mixture thereof. In still another embodiment, the coating material may comprise an edible wax. Edible waxes may be derived from animals, insects, or plants. Non-limiting examples include beeswax, lanolin, bayberry wax, carnauba wax, and rice bran wax. Tablets and pills may additionally be prepared with enteric coatings.

Alternatively, powder's or granules embodying the compositions disclosed and made obvious herein may be incorporated into a food product. The food product may be a drink. Non-limiting examples of a suitable drink include fruit juice, a fruit drink, an artificially flavored drink, an artificially sweetened drink, a carbonated beverage, a sports drink, a liquid diary product, a shake, and so forth. The compositions may also be in liquid dosage forms for oral administration. Liquid dosage forms include aqueous and nonaqueous solutions, emulsions, suspensions and solutions and/or suspensions reconstituted from non-effervescent granules, containing suitable solvents, preservatives, emulsifying agents, suspending agents, diluents, sweeteners, coloring agents, and flavoring agents.

Combining of Nutrition with other Modalities

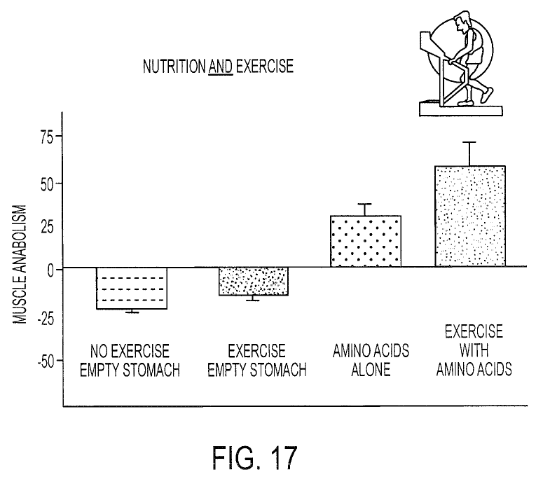

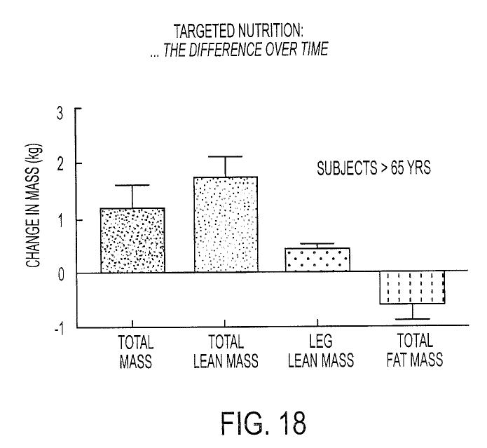

FIGS. 17 and 18 present the improved outcome as a result of combining therapeutic modalities of exercise and specific nutritional support. While exercise alone is able to stimulate and increase protein synthesis and functional outcomes, when therapeutic nutrition use is added and significant improvement is observed in both synthetic rates and practical outcomes. These outcomes demonstrate the superior approach of each method of care as well as the combined benefits of both uses in the elderly.

The adaptation of the methods and product formula described herein can be combined with use of drug therapy medications targeting sarcopenia and other muscle wasting conditions (improve muscle mass and weight gain) and improvement of cardiovascular disease (decreased triglycerides, cholesterol and blood pressure) and co-management of inflammatory based conditions (COPD, diabetes and cognitive decline).

The compositions of the invention may be utilized in methods to increase muscle mass, strength and physical function. In an embodiment, the method comprises administering the composition as described above twice per day between meals. The amount per dose may be about 6, 7, 8, 9, 10, 11, 12, 13, 14, 15, 16, 17, 18, 19, 20, 21, 22, 23, 24, 25, 26, 27, 28, 29, or 30 g. Alternatively, the composition may be administered one day per day, two, three times per day, or four times per day.

In an alternative or further embodiment of a method of delivery, the composition may also be used in conjunction with exercise. For example, the composition may given before or immediately after exercise.

While the invention has been disclosed in connection with certain preferred embodiments, this should not be taken as a limitation to all of the provided details. Modifications and variations of the described embodiments may be made without departing from the spirit and scope of the invention, and other embodiments should be understood to be encompassed in the present disclosure as would be understood by those of ordinary skill in the art.

1. A composition comprising:
a therapeutic component including:
intact protein; and
at least one essential amino acid selected from the group consisting of:
leucine, isoleucine, valine, lysine, methionine, histidine, phenylalanine, theonine, and tryptophan; and
a carrier component comprising a fruit juice.

2. The composition of claim 1 wherein said carrier component comprises a combination of fruit juices.

3. The composition of claim 2 wherein said combination comprises a combination selected from the group consisting of: loquat and lime, mango and peach, black cherry and apple, pomegranate and cranberry, plum and tea, apple and peach, blueberry and grape, cranberry and lemon, orange and naranjilla, and pineapple and naranjilla.

4. The composition of claim 1 further comprising a low-glycemic carbohydrate.

5. The composition of claim 1 further comprising an omega-3 fatty acid.

6. The composition of claim 1 further comprising at least one of arginine or citrulline.

7. A drink product comprising:
a liquid solution comprising:
protein;
at least one essential amino acid; and
a fruit juice; and
a bottle for holding said liquid solution, said bottle having:
a mouth;
a neck arranged toward said mouth;
and a bottom having a larger circumference than said mouth.

8. The drink product of claim 7 wherein said bottle holds between about 8 and about 12 ounces of liquid.
